(19) United States
(12) Patent Application Publication
(10) Pub. No.: US 2025/0193609 A1
(43) Pub. Date: Jun. 12, 2025

(54) HEARING INSTRUMENT AND METHOD FOR OPERATING THE HEARING INSTRUMENT

(71) Applicant: Sivantos Pte. Ltd., Singapore (SG)

(72) Inventor: Kunibert Husung, Erlangen (DE)

(21) Appl. No.: 18/976,614

(22) Filed: Dec. 11, 2024

(30) Foreign Application Priority Data
Dec. 12, 2023 (DE) 10 2023 212 514.5

(51) Int. Cl. H04R 25/00; A61B 5/38; G01D 5/241

(52) U.S. Cl. CPC H04R 25/305; A61B 5/38; G01D 5/241; H04R 25/505; H04R 25/604; H04R 25/609; H04R 25/656; H04R 2225/0216

(57) ABSTRACT

A hearing instrument has a housing to be worn in a designated wearing position behind the ear, an earpiece to be inserted into the auditory canal of a user, and a flexible connector that connects the earpiece to the housing. The hearing instrument further includes an output transducer for converting an output audio signal into a sound signal to be output to the user and a capacitive sensor with a control and evaluation circuit and two sensor electrodes electrically connected thereto. One of the sensor electrodes is arranged in the housing, while the other sensor electrode is arranged in the connector and/or in the earpiece.

### CROSS-REFERENCE TO RELATED APPLICATION

This application claims the priority, under 35 U.S.C. § 119, of German Patent Application DE 10 2023 212 514.5, filed Dec. 12, 2023; the prior application is herewith incorporated by reference in its entirety.

### FIELD AND BACKGROUND OF THE INVENTION

The invention relates to a hearing instrument having a housing to be worn in a designated wearing position behind the ear of a user, an earpiece to be inserted into the auditory canal of an ear, and a flexible connector. Further, the invention relates to a method of operating such a hearing instrument.

A hearing instrument generally refers to an electronic device that assists a person wearing the hearing instrument (who is referred to as “wearer” or “user” below) with the ability to hear. In particular, the invention relates to hearing instruments that are configured to fully or partly compensate for a hearing loss of a hearing-impaired user. Such a hearing instrument is also referred to as “hearing aid.” Additionally, there are hearing instruments that protect or improve the ability of users with normal hearing to hear, for example enable improved speech comprehension in complex hearing situations. Hearing instruments furthermore also include wireless headphones (worn in or on the ear), in particular so-called ear plugs and headsets.

Hearing instruments in general, and specifically hearing aids, are usually embodied to be worn on the head of the user and, in particular, in or on an ear in this case, in particular as behind-the-ear devices (BTE devices) or in-the-ear devices (ITE devices). In terms of their internal structure, hearing instruments regularly comprise at least one (acousto-electric) input transducer, a signal processing unit (signal processor), and an output transducer. During the operation of the hearing instrument, the input transducer or each input transducer records airborne sound from the surroundings of the hearing instrument and converts the airborne sound into an input audio signal (i.e., an electrical signal that transports information about the ambient sound). The input audio signal or each input audio signal is processed in the signal processing unit (i.e., modified in terms of its sound information) in order to assist the ability of the user to hear, in particular to compensate for a loss of hearing of the user. The signal processing unit outputs an appropriately processed audio signal to the output transducer. In addition or as an alternative to frequency-dependent amplification of the input audio signal, signal processing in modern hearing instruments regularly encompasses a multiplicity of other functions, e.g., beamforming (i.e., direction-dependent damping), active noise suppression, wind noise suppression, feedback damping, binaural processing to support spatial hearing, dynamic and/or spectral compression, etc.

In most cases, the output transducer is in the form of an electro-acoustic transducer, which converts the (electrical) output audio signal back into airborne sound, wherein this airborne sound—which is being modified in relation to the ambient sound—is output into the auditory canal of the user. In the case of a hearing instrument worn behind the ear, the output transducer, which is also referred to as “receiver”, is usually integrated in a housing of the hearing instrument outside of the ear. The sound output by the output transducer is guided into the auditory canal of the user by means of a sound tube in this case. In an alternative thereto, the output transducer can also be arranged in the auditory canal, and consequently external of the housing worn behind the ear. Such BTE devices are also referred to as RIC devices (from the term “receiver in canal”). Hearing instruments that are worn in the ear and are dimensioned to be so small that they do not protrude beyond the auditory canal to the outside are also referred to as CIC devices (from the term “completely in canal”).

In further embodiments, the output transducer can also be in the form of an electromechanical transducer, which converts the output audio signal into structure-borne sound (vibrations). The structure-borne sound may, for example, be output to the cranial bone of the user.

A “hearing system” generally refers to an arrangement of devices and optionally other structures that provide functions for operating a hearing instrument. In the simplest case, the hearing system consists only of the hearing instrument itself. As a rule, however, in addition to the hearing instrument, the hearing system comprises at least one peripheral device that interacts with the hearing instrument when the latter is in operation, e.g., a further hearing instrument for the other ear of the user, a remote control, an external microphone, a programming device for the hearing instrument or a charger. In addition to the hearing instrument, modern hearing systems often also provide a software application that can be installed on a computer (especially a portable computer), e.g., a smartphone or a tablet computer, and that contributes functions to the operation of the hearing instrument (e.g., remote control, programming, firmware updates, data backup, complex signal processing and/or an Internet connection). The computer on which this software application (hereinafter referred to as “hearing app”) is installed is also a peripheral device of the hearing instrument, which is data-connected to the hearing instrument, when the hearing instrument is in operation. However, the computer is not normally part of the hearing system itself, insofar as it is manufactured and distributed independently of the components of the hearing system and can be used for a multiplicity of other applications not connected to the hearing system. Rather, the computer (i.e., the user's smartphone or tablet computer in particular) is used by the hearing app only as an external resource for computing power, storage space and communication services.

The proper functioning of a hearing instrument, and thus the benefit that the hearing instrument brings to its wearer in daily operation, depends crucially on the fit of the housing and earpiece on or in the wearer's ear. In other words, the hearing instrument can only satisfactorily fulfill the task intended therefor if the housing and the earpiece are arranged in the respective designated wearing position behind or in the ear.

Firstly, this is because the signal processing of the hearing instrument is calibrated to a specific acoustic situation at the location of the microphone or each microphone of the hearing instrument, e.g., to a certain alignment of the (respective) microphone with respect to the head and to certain acoustic shadowing of the microphone by the ear. If the housing of the hearing instrument is worn behind the ear in a manner that deviates from the designated wearing position, then this changes the acoustic situation to which the microphone is exposed, and hence also the input audio signal recorded by the microphone-if there is ambient noise. Signal processing calibrated for a different acoustic situation can no longer optimally handle the input audio signal recorded by the mispositioned microphone. For example, this can manifest itself in that misaligned beamforming leads to damping the used signal (e.g., the voice of a conversation partner) wanted by the user rather than an unwanted background noise, in that active noise suppression no longer functions satisfactorily, etc.

An undesirable effect of mispositioning of the earpiece can be a reduced acoustic sealing of the auditory canal by the earpiece, and this can lead to undesirable side effects, e.g., increased noise levels or the occurrence of acoustic feedback. In the case of a BTE device, misalignment of the housing can also cause such effects, for example if the connector is under mechanical stress due to the mispositioning of the housing or is bent too much. In this case, the connector can exert a mechanical load on the earpiece, which gradually pulls the earpiece completely or partially out of the ear or otherwise deflects from the correct fit in the auditory canal.

### SUMMARY OF THE INVENTION

Against this background, the problem addressed by the invention is that of enabling an improved check for the correct fit of a hearing instrument.

With the above and other objects in view there is provided, in accordance with the invention, a hearing instrument, comprising:
a housing to be worn in a designated wearing position behind an ear of a user;
an earpiece to be inserted into an auditory canal of the ear and a flexible connector that connects the earpiece to the housing;
an output transducer for converting an output audio signal into a sound signal to be output to the user; and
a capacitive sensor having a control and evaluation circuit and two sensor electrodes electrically connected to the control and evaluation circuit;
wherein one of the two sensor electrodes is arranged in the housing and another of the two sensor electrode is arranged in at least one of the connector or the earpiece.

In other words, the invention is based on a hearing instrument having a housing that should be worn in a designated wearing position behind the ear of a user. The hearing instrument additionally has an earpiece to be placed in the ear of the user, a flexible connecting part that connects the housing and the earpiece, and an output transducer for converting an (electrical) output audio signal into a sound signal to be output to the user. The output transducer is preferably formed by an electro-acoustic converter, i.e., a loudspeaker (“receiver”). The hearing instrument is therefore a BTE (behind-the-ear) device, to be precise either a conventional hearing instrument having an output transducer arranged in the housing or an RIC (receiver-in-canal) device in which the output transducer is arranged in the earpiece. In the former case, the connector is formed by a sound tube, which supplies the sound produced by the output transducer to the earpiece. In the latter case, the connector comprises an electrical connecting cable by means of which the output audio signal is supplied to the output transducer which is arranged in the earpiece.

In all embodiments described above, the hearing instrument also comprises a capacitive sensor that comprises a control and evaluation circuit (hereinafter also referred to as “sensor controller”) and (at least) two sensor electrodes electrically connected thereto. According to the invention, one of the two sensor electrodes is arranged in the housing and the other sensor electrode is arranged in the connector and/or the earpiece.

The capacitive sensor enables detection of generally dielectric or electrically conductive structures in the vicinity of the sensor, specifically body structures such as e.g., the head and ear of the user in particular. What is exploited here is the fact that, on account of the variety of surrounding body structures of the user, the (capacitive) sensor signal output by the capacitive sensor depends in a characteristic manner on the fit of the housing and the connecting part and, optionally, the earpiece on or in the ear. In other words, the sensor signal output by the capacitive sensor changes in a characteristic manner when the user displaces the hearing instrument (in particular the housing of the same) relative to the ear. It was recognized that the correct fit of the hearing instrument in the designated wearing position can be recognized on the basis of this dependence of the capacitive sensor signal. Allocating the two sensor electrodes of the capacitive sensor to the housing on the one hand and to the connecting part and/or the earpiece on the other hand turned out to be particularly advantageous in this context since this arrangement of the sensor electrodes allows a particularly precise and effective recognition of a possible mispositioning of the hearing instrument. In particular, this allows effective determination as to whether the connector runs close to the ear when the hearing instrument is inserted as envisaged, whence the position of the housing and the earpiece can be deduced in turn.

The control and evaluation circuit (sensor controller) of the capacitive sensor is preferably configured to apply an alternating voltage (also referred to as “(capacitive) sensor voltage”) to at least one of the sensor electrodes and measure a response signal created under the action of this alternating voltage, the response signal being characteristic of an electrical capacitance associated with the sensor electrode. The sensor controller is preferably embodied as a microcontroller. In this case, the functionality of the sensor controller is implemented as software. However, the sensor controller can also be embodied as a non-programmable (analog or digital) electrical circuit within the scope of the invention. Within the scope of the invention, the sensor controller in both cases either can be embodied as a separate component (e.g., as a separate integrated circuit) or can be integrated in a larger structural unit together with other functions.

In order to rule out possible disturbances of the sound signal output by the output transducer by the capacitive sensor, the sensor controller is preferably configured to output the sensor voltage as alternating voltage at a frequency that exceeds the audible frequency spectrum, in particular not below 20 KHz. In the preferred embodiment, the frequency of the sensor voltage is between 20 kHz and 10 MHz, in particular between 20 kHz and 100 kHz, e.g., at 60 KHz.

In principle, the capacitive sensor can be based on one of two inherently conventional functional principles within the scope of the invention.

According to a first functional principle, referred to as e.g., “single-electrode measurement” or “self-capacitance measurement” (self-capacitance sensing), the sensor controller measures the response signal at the same sensor electrode to which it applies the sensor voltage. The sensor electrodes of the capacitive sensor are activated independently of one another by the sensor controller using the sensor voltage. As a capacitance-dependent response signal, the sensor controller measures at each sensor electrode, for example, the current flowing under the effect of the sensor voltage on this sensor electrode or the frequency of the sensor voltage (wherein the fact that the respective sensor electrode is part of a resonant circuit with a resonance that depends on the capacitance is exploited). In all cases, the response signal here is characteristic of the (electrical) capacitance of each sensor electrode in relation to an external ground potential. In the “single-electrode measurement”, the external ground potential formed here e.g., by the body of the user acts as a counter-electrode of the capacitor whose capacitance is measured by the sensor.

According to a second functional principle, referred to as “two-electrode measurement”, “transmitter/receiver principle” or “relative capacitance measurement” (mutual capacitance sensing), the sensor controller applies the sensor voltage to a first sensor electrode (transmitting electrode) and measures the response signal at the other sensor electrode (receiving electrode). As a capacitance-dependent response signal, the sensor controller in this case measures, for example, the current created under the effect of the sensor voltage via an electrical field in the receiving electrode. In this case, the response signal is characteristic of the (electrical) capacitance of the capacitor formed by the two sensor electrodes. In the “two-electrode measurement”, the body of the user acts as a disturbance potential which changes the capacitance of the capacitor formed by the two sensor electrodes and is detected thereby.

The capacitive sensor of the hearing instrument according to the invention is preferably embodied as a “relative-capacitance sensor” (mutual capacitance sensor) since the sensor controller can be implemented particularly easily from a circuitry point of view in this embodiment; in a particularly simple embodiment, the sensor controller in this sense comprises a single-channel evaluation circuit, i.e., comprises only a single sensor input for measuring a single response signal.

As mentioned above, the hearing instrument in preferred embodiments is embodied as an RIC device having an output transducer (in particular an electro-acoustic output transducer) arranged in the earpiece. The electrical connecting cable contained in the connector, via which the output audio signal is supplied to the output transducer, is preferably used as one of the sensor electrodes of the capacitive sensor in this case. Within the scope of the invention, the electrical signal lines of the connecting cable, via which the output audio signal is supplied to the output transducer, can also be used as a sensor electrode of the capacitive sensor in this case. In addition to the signal lines, the connecting cable contains a separate electrical conductor used (in particular exclusively) as a sensor electrode in an alternative. In addition or as an alternative to the use of the connecting cable as a sensor electrode, the output transducer (in particular a metallic housing of the output transducer) is used as one of the sensor electrodes of the capacitive sensor. In an expedient configuration, the connecting cable and the output transducer are short-circuited to each other in order to form a single sensor electrode; in particular, the sensor electrode is formed by a ground conductor or a shield conductor of the connecting cable, which is short-circuited to the metallic housing of the output transducer. Within the scope of the invention, however, it is alternatively also conceivable that the connecting cable and the output transducer are used as different, in particular independently activated, sensor electrodes in the framework of the capacitive sensor.

In further embodiments of the invention, the hearing instrument—as also mentioned above—is embodied as a conventional BTE device, wherein the output transducer is arranged in the housing. In this case, the connector is embodied as a hollow sound tube which serves to conduct the sound signal created by the output transducer-always an electro-acoustically embodied output transducer in this case—to the earpiece. One of the sensor electrodes of the capacitive sensor is arranged in this sound tube, e.g., in the form of a wire or wire mesh or wire netting guided in the sound tube or embedded in the wall of the sound tube or in the form of an electrically conductive film or coating applied to the inner wall of the sound tube.

A further embodiment of the invention relates to a hearing system that comprises the hearing instrument according to the invention (in particular in one of the variants described above) and a fit-monitoring unit. In this case, the fit-monitoring unit is configured to check the fit of the hearing instrument on the ear of the user on the basis of a sensor signal output by the capacitive sensor. The fit-monitoring unit, implemented either as a software module or as a non-programmable electronic circuit, preferably performs this check by comparing the sensor signal output by the capacitive sensor with a stored reference value. Since the shape of the head and ear is individual to each user, the reference value is also preferably determined on an individual basis in this case for the respective user. In an alternative embodiment, the fit-monitoring unit comprises a neural network, which is supplied with the sensor signal of the capacitive sensor and which is trained, in particular on an individual basis, for the respective user in order to check the fit of the hearing instrument on the basis of the sensor signal. The fit-monitoring unit is preferably integrated in the hearing instrument. In this case, the hearing system can also consist only of the hearing instrument itself, which is equipped with the fit-monitoring unit. In an alternative, the fit-monitoring unit is located outside the hearing instrument, e.g., in a peripheral device or software application (hearing app) of the hearing system.

The fit-monitoring unit is also configured to cause a message to be output to inform the user of the deviating fit of the housing should the fit be determined as deviating from the designated wearing position. This message is preferably displayed by outputting a text message or a visual cue (e.g., in the form of a schematic illustration of the hearing instrument housing arranged on the ear) on a peripheral device, e.g., on the display of a smartphone of the user by the hearing app associated with the hearing instrument. In addition to that or in an alternative, the message is output by the hearing instrument itself, e.g., in the form of a signal tone, a voice message output via the receiver of the hearing instrument or by a tactile signal (e.g., vibration).

In a further embodiment, the fit-monitoring unit is configured to adjust at least one signal processing parameter of the hearing instrument on the basis of the capacitive sensor signal output by the capacitive sensor, in order to adapt the signal processing (i.e., the function of the signal processor) of the hearing instrument to the fit of the housing. In particular, the fit-monitoring unit adapts the alignment of a beamformer of the hearing instrument in such a way that a determined deviation of the fit of the housing with respect to the designated wearing position is compensated.

With the above and other objects in view there is also provided, in accordance with the invention, a method that uses the hearing instrument according to the invention in one of the embodiments described above. The statements made above in relation to embodiments and component parts of the hearing instrument and the associated effects and advantages therefore also apply mutatis mutandis to corresponding embodiments of the method, and vice versa.

In the course of the method according to the invention, a capacitive sensor signal is ascertained as a measure for the fit of the housing and the connecting part on the ear of the user (and optionally the fit of the earpiece in the ear of the user) using the sensor electrodes of the capacitive sensor of the hearing instrument according to the invention allocated to the housing and the connecting part and/or the earpiece. A message indicating the deviating fit of the hearing instrument is output should the fit be determined as deviating from the designated wearing position of the housing or the connector and/or the earpiece. In addition to that or in an alternative, at least one signal processing parameter of the hearing instrument is adjusted on the basis of the capacitive sensor signal output by the capacitive sensor, in order to adapt the signal processing of the hearing instrument to the fit of the hearing instrument.

Other features which are considered as characteristic for the invention are set forth in the appended claims.

Although the invention is illustrated and described herein as embodied in a hearing instrument and method for operating same, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawing figures.

### BRIEF DESCRIPTION OF THE FIGURES

FIG. 1 shows a schematic illustration of a hearing system having a hearing instrument and a software application (hearing app) installed on a smartphone of the user, wherein the hearing instrument comprises a housing that is wearable behind an ear of a user, an earpiece that should be inserted into the auditory canal of the user and comprises an electro-acoustic output transducer (receiver) integrated therein and a flexible connecting part mechanically and electrically connecting the housing to the earpiece, and wherein the hearing instrument comprises a capacitive sensor that comprises a first sensor electrode arranged in the housing and uses a connecting cable guided in the connecting part as a second sensor electrode,

FIG. 2 shows a schematic illustration of an embodiment variant of the hearing instrument, in which the sensor electrodes of the capacitive sensor are operated as a transmitting or receiving electrode; and

FIG. 3 shows, in an illustration as per FIG. 2, a further embodiment variant of the hearing instrument, in which the sensor electrodes of the capacitive sensor are operated as independently independent sensor electrodes.

Parts and quantities corresponding to one another are always provided with the same reference signs in all figures.

### DETAILED DESCRIPTION OF THE INVENTION

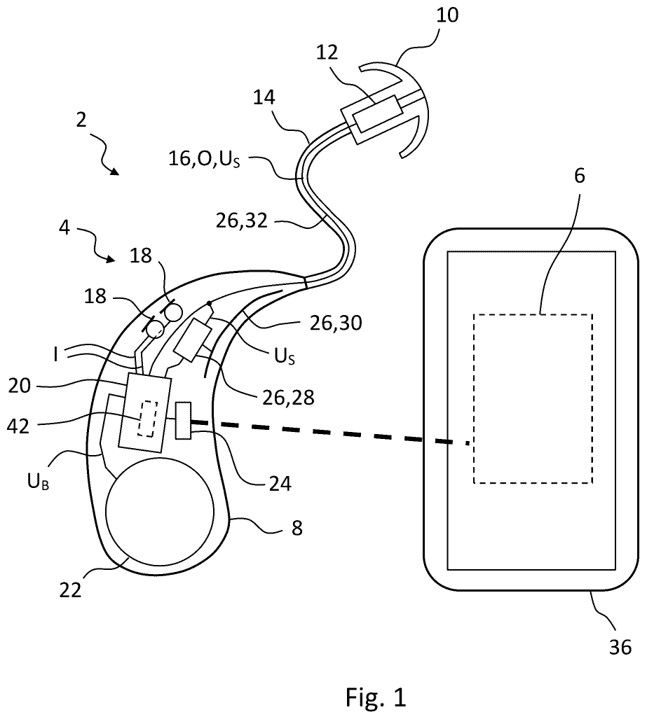

Referring now to the figures of the drawing in detail and first, in particular, to FIG. 1 thereof, there is shown a crude schematic illustration of a hearing system 2 that is formed from a hearing instrument 4 and an associated software application (hearing app 6).

By way of example, the hearing instrument 2 is a hearing aid, i.e., a hearing instrument configured to assist a hearing-impaired user with the ability to hear. In the embodiment depicted here, the hearing instrument 2 is embodied as an RIC device. Accordingly, it comprises a housing 8 worn behind an ear of a user when used as intended and an earpiece 10 inserted into the auditory canal of the user when used as intended, wherein an electro-acoustic output transducer (receiver 12) is integrated in the earpiece 10. The hearing instrument 2 furthermore comprises a flexible connector 14 that mechanically connects the housing 8 to the earpiece 10. In the case of the RIC device depicted in FIG. 1, the connector 14 comprises an electrical connecting cable 16 for the receiver 12.

The hearing instrument 4 comprises the following components within its housing 8:
at least one microphone 18 (two microphones 18 in the illustrated example), forming an input transducer,
a signal processor 20 (in particular a digital signal processor, DSP),
a battery 22,
a wireless communications apparatus 24 having—not depicted explicitly—an RF antenna and a transceiver unit electrically connected thereto, and
a capacitive sensor 26 formed from a control and evaluation circuit (sensor controller 28) and a plurality (two in the embodiment shown) of sensor electrodes 30 and 32 (with one being inside the housing 8 and one in the connection to the earpiece 10 or in the earpiece).

During normal operation of the hearing instrument 4, the microphones 18 each record airborne sound from the surroundings of the hearing instrument 4. The microphones 18 convert the sound into an (input) audio signal I, i.e., into an electrical signal that contains information about the recorded sound. The respective input audio signal I is supplied within the hearing instrument 4 to the signal processor 20, which modifies this input audio signal I to assist the ability of the user to hear. In a specific case, the signal processor 20 amplifies the input audio signal in a frequency-selective manner to compensate for a hearing loss of the user.

The signal processor 20 outputs an output audio signal O to the receiver 12 via signal lines of the electrical connecting cable 16 that is guided in the housing 8 and the connector 14. The output audio signal O is an electrical signal that contains information about the processed and hence modified sound. The signal processor 20 and all other electrical or electronic components of the hearing instrument 4 are supplied from the battery 22 with a DC voltage referred to as operating voltage U_{B}.

The wireless communications apparatus 24 is used for (wireless) data exchange between the hearing instrument 4 and the hearing app 6 and/or optionally further components of the hearing system 2, e.g., a further hearing instrument 4 (not shown) for the other ear of the user.

The hearing app 6 serves in particular to remotely control and program the hearing instrument 4. In the operation of the hearing system 2, the hearing app 6 is installed in executable fashion on a computer (in particular a portable computer). In the example shown, this computer is a smartphone 36 of the user. In this case, the computer itself, in particular the smartphone 36, is not a constituent part of the hearing system 2 but is used by the hearing app 6 only as an external resource for computing power, storage space and communication services. In particular, the hearing app 6 accesses a wireless communications apparatus (not shown in detail) of the smartphone 36 in order to exchange data with the hearing instrument 4.

The wireless communications apparatus 24 of the hearing instrument 4 and the wireless communications apparatus of the smartphone 36 are generally embodied for the exchange of radio signals (also: radio waves, specifically radiofrequency electromagnetic radiation above 100 MHZ). Preferably, the data transfer between the hearing instrument 4 and the smartphone 36 (and hence the hearing app 6) is based on the Bluetooth standard, at a radiofrequency of 2.4 GHz.

The capacitive sensor 26 serves to detect the fit of the hearing instrument 2 on the ear of the user, i.e., to check whether the housing 8 is in a designated wearing position behind the ear of the user or in a position deviating therefrom. In this case, a first sensor electrode 30 of the capacitive sensor 26 is formed by a one- or two-dimensional electrically conductive structure, which is arranged in the housing 8, in particular adjacent or in proximity to an inner side of the housing wall. For example, the first sensor electrode 30 is formed by a wire conductor, a wire mesh or netting, a metal stamped part, a foil or an electrically conductive coating applied to the inner side of the housing wall. The second sensor electrode 32 is formed by the portion of the connecting cable 16 guided in the connecting part 14, in this case either by one of the signal conductors or both signal conductors or by an optionally available separate conductor of the connecting cable 16.

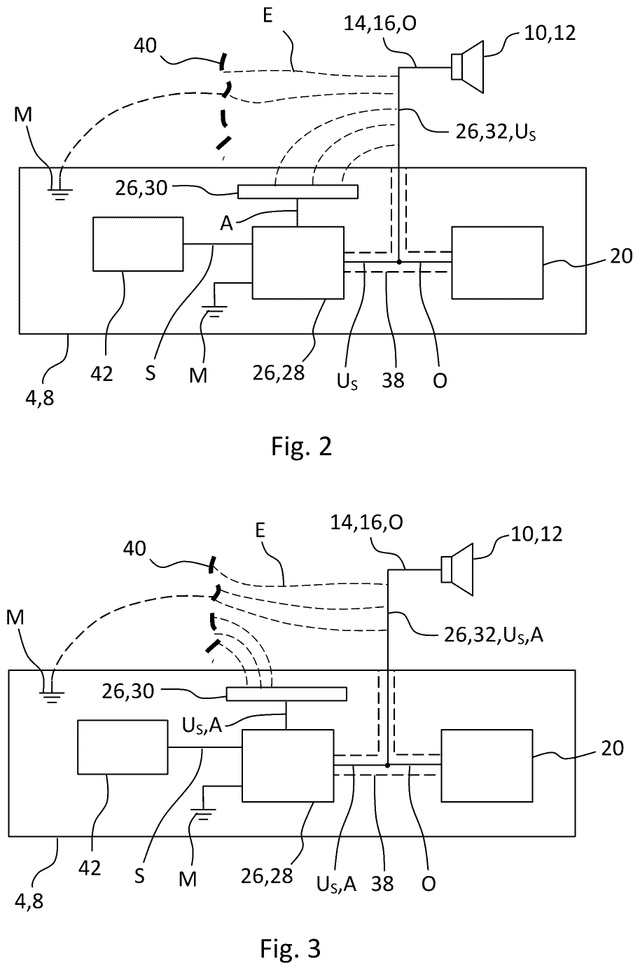

In order to prevent a capacitive interaction between the sensor electrode 30 and the portion of the connecting cable 16 that is guided in the interior of the housing 8, the housing inner portion of the connecting cable 16 is preferably electrically shielded from the sensor electrode 30 by a shield 38 (FIGS. 2 and 3). The electrical supply lines between the sensor controller 28 and the sensor electrodes 30 and 32, too, are preferably likewise electrically shielded. Shielding 38 is optionally designed as a potential-controlled shield (“driven shield”).

The sensor controller 30 controls—as explained in more detail below—at least one of the sensor electrodes 30 and 32 (the connecting cable 16 used as sensor electrode 32 in the example as per FIG. 1) with an AC voltage referred to as sensor voltage U_{S}, whose AC voltage frequency is between 20 KHz and 10 MHz and for example is 60 KHz. The sensor voltage U_{S} is thus between and spaced apart from, in particular spectrally spaced apart from, the operating voltage U_{B} produced by the battery 22 on the one hand and the radiofrequency of the wireless communications apparatus 24 on the other hand. It is also above the audible frequency spectrum, and so perceptible disturbances of the output audio signal O and the sound signal created therefrom due to sensor voltage U_{S} being fed into the connecting cable 16 are precluded.

The signal processor 20, the transceiver of the wireless communications apparatus 24 and the sensor controller 28 are each formed either by a programmable circuit (e.g., a microprocessor) with software installed thereon or by a non-programmable circuit (e.g., in the form of an ASIC) or by a combination of at least one programmable subunit and at least one non-programmable subunit. As indicated by way of example in FIG. 1, the signal processor 20, the communications apparatus 24 and the sensor controller 28 can be formed as separate circuits in each case. In an alternative, the communications apparatus 24 and/or the sensor controller 28 with the signal processor 20 and/or at least one optionally present further control unit of the hearing instrument 4 are integrated in a common circuit.

In two alternative embodiment variants of the hearing instrument 4 as per FIGS. 2 and 3, the sensor electrodes 30 and 32 are activated differently by the sensor controller 28.

In the embodiment as per FIG. 2, the sensor electrodes 30 and 32 are operated by the sensor controller 28 as transmitting electrode and receiving electrode. In this case, the sensor controller 28 applies the sensor voltage U_{S} to the sensor electrode 32 (i.e., the connecting cable 16). Under the action of the sensor voltage U_{S}, the sensor electrode 32 creates an electric field E that alternates at the AC voltage frequency in a surrounding spatial volume and causes a charge displacement in the sensor electrode 30, and hence an electric current flow. The sensor controller 30 preferably measures the current intensity of this current flow at the sensor electrode 30 as response signal A, said response signal A being characteristic of the (electrical) capacitance of the capacitor formed by the sensor electrodes 30 and 32. The capacitive sensor 26 is thus embodied as a “relative capacitance sensor” (mutual capacitance sensor).

Body structures 40 of the user, e.g., the head and the ear, which are arranged in proximity to the housing 8 (and schematically indicated in FIG. 4) and on which the housing 8 and the connector 14 are worn act as a disturbance potential on account of the AC conductivity of the human body and the electrical connection of the body to the sensor controller 28 via ground M, said disturbance potential reducing the capacitance of the capacitor formed by the sensor electrodes 34a and 34b, and thus the value of the measured response signal A, to an ever greater extent, the larger the body structures are and the closer they are to sensor electrodes 30 and 32.

In the embodiment as per FIG. 3, the sensor electrodes 30 and 32 are operated by the sensor controller 28 as separate (i.e., mutually independent) sensor electrodes. In this case, the sensor controller 28 applies the sensor voltage U_{S} to both sensor electrodes 30 and 32 and measures the response signal A, created under the action of the sensor voltage U_{S}, independently for each of the sensor electrodes 30 and 32; in this case, too, the current intensity of the current flowing to the respective sensor electrode 30, 32 is preferably measured as response signal A. In this case, the response signal A associated with the respective sensor electrode 30, 32 is characteristic of the capacitance of the respective sensor electrodes 30 and 32 with respect to ground M. The capacitive sensor 26 is therefore embodied as a “self-capacitance sensor” in this embodiment. The sensor voltage U_{S} is preferably applied to the two sensor electrodes 30 and 32 in phase, and so no electric field E extends between the two sensor electrodes 30 and 32 themselves.

The electric field E created by the respective sensor electrode 30, 32 under the action of the sensor voltage U_{S} in this case extends between the respective sensor electrode 30, 32 and the respective nearest body structures 40 of the user. The body structures 40, in particular the head and the adjacent ear, of the user located in the vicinity of the hearing instrument 4 and at ground M for AC voltages act as a counter electrode for the respective sensor electrode 30, 32 in this embodiment of the capacitive sensor 26. In other words, each of the sensor electrodes 30, 32 forms the capacitor, the capacitance of which is measured at the respective sensor electrode 30, 32, together with the nearest body structures 40. This capacitance, and hence the value of the measured response signal A,—unlike in the embodiment as per FIG. 2—becomes ever higher the larger the body structures 40 are, and the closer they are arranged to the respective sensor electrode 30, 32.

The embodiment as per FIG. 2 is advantageous over the embodiment as per FIG. 3 in that the sensor controller 28 manages with a single sensor input for measuring the one response signal A in this case and can therefore be realized particularly easily.

In the embodiment as per FIGS. 2 and 3, the capacitive sensor 26 only detects the fit of the housing 8 and connector 14 directly since the sensor electrodes 30 and 32 are arranged only in the region of these parts of the hearing instrument 4. The fit of the earpiece 10 in the ear of the user is only checked indirectly in this case, the assumption being made that the earpiece 10 is correctly positioned in the ear of the user when the position of the housing 8 and connector 14 corresponds to the designated wearing position.

In a further embodiment of the hearing instrument 4 (not illustrated in detail), a metallic housing of the receiver 12 is also used as a sensor electrode of the capacitive sensor 26, in addition to the connecting cable 16. In this case, the housing of the receiver 12 is preferably short-circuited with the conductor of the connecting cable 16 (in particular a shielding of the connecting cable 16) used as sensor electrode 32, and so the connecting cable 16 and the receiver 12 together form the sensor electrode 32. In this case, the capacitive sensor 26 also detects the (correct or incorrect) fit of the earpiece in the ear of the user directly.

In an alternative embodiment, the receiver 12 (more precisely, the metallic housing of the receiver 12) is operated by the sensor controller 28 as a separate sensor electrode, in addition or as an alternative to the connecting cable 16. This separate sensor electrode is used to detect the fit of the earpiece in the ear of the user independently of the fit of the housing 8 and connector 14 on the ear.

In all embodiments described above, the measured response signal A or each measured response signal is used in the operation of the hearing instrument 4 in a method for checking the fit of the hearing instrument 4. In order to automatically carry out this method, the hearing system 2 comprises a fit-monitoring unit 42 that is configured to use a sensor signal S output by the capacitive sensor 26 to check the fit of the housing 8 on the ear of the user, i.e., to determine whether the hearing instrument 4 (i.e., housing 8, the connector 14 and optionally the earpiece 10) is in a designated wearing position on the ear of the user or whether the fit of the housing 8, of the connector 14 and optionally of the earpiece 10 deviates from the designated wearing position.

Within the scope of the invention, the fit-monitoring unit 42 might be formed by a non-programmable electronic circuit, e.g., in the form or as part of an ASIC. Preferably, however, the fit-monitoring unit 42 is formed by a software module which, for example, is installed in executable fashion on the signal processor 20 (see FIG. 1) or optionally another programmable control circuit of the hearing instrument 4. In a further embodiment of the invention, the fit-monitoring unit 42 is implemented as part of the hearing app 6 outside the hearing instrument 4.

The sensor signal S output from the sensor controller 28 to the fit-monitoring unit 42 contains the (unchanged) value of the or each response signal A or a quantity derived from it, for example, the capacitance assigned to the sensor electrodes 30, 32 (optionally each) or an inverted or scaled quantity.

In order to monitor the fit of the hearing instrument 4 behind the ear of the user, the fit-monitoring unit 42 compares the sensor signal S in a preferred configuration of the method with a stored reference value which reproduces the value of the sensor signal S when the hearing instrument 4 (i.e., the housing 8 and the connector 14 in particular) is positioned in the designated wearing position. Should the fit-monitoring unit 42 determine a significant deviation of the sensor signal S from the reference value (in particular a deviation that exceeds a predetermined tolerance value), it causes the output of a (warning) message to the user, which informs the user of the incorrect fit (i.e., a deviation from the designated wearing position) of the hearing instrument 4.

Since the value of the sensor signal S is also influenced by the approach of further body parts to the hearing instrument 4, e.g., the approach of a hand or a finger, but these disturbing influences are usually only short-lived, the fit-monitoring unit 42 is preferably embodied to only trigger the output of the warning message if the sensor signal S continuously deviates significantly from the reference value for longer than a predetermined time interval (e.g., more than 2 min).

For example, the warning message is in the form of:
a beep output into the ear of the user via the receiver 12 or output by the hearing app 6 via a speaker of the smartphone 36,
a voice message output into the ear of the user via the receiver 12 or output by the hearing app 6 via a speaker of the smartphone 36,
a text message or graphic displayed by the hearing app 6 on the display of the smartphone 36, or
a tactile alarm (e.g., a vibration signal) output via the hearing instrument 4 or the smartphone 36, or
a combination of the above notification methods.

The reference value is preferably learned on an individual basis when the hearing instrument 4 is adapted to the user (during the fitting process), e.g., by virtue of the sensor signal S being captured and stored as a reference value after the hearing instrument 4 was positioned in the correct designated wearing position behind the ear of the user by a specialist.

In a developed embodiment of the invention, it is not only a single reference value for the sensor signal S that is stored in the fit-monitoring unit 42 but a function or characteristic curve or specific value table, which in addition to the characteristic specific value for the designated wearing position also contains specific values for deviating positions of the hearing instrument 4 behind the ear. For example, the function, characteristic curve or specific value table is likewise ascertained when the hearing instrument is adapted to the user, by virtue of the housing 8 being placed at different positions behind the ear by a specialist, and by virtue of the respective value of the sensor signal S being captured and stored in each position together with a position specification. According to the method, by comparing the current value of the sensor signal S with the function, characteristic curve or specific value table in the operation of the hearing instrument 4, the fit-monitoring unit 42 in this embodiment determines whether the seat of the hearing instrument 4 corresponds to the designated wearing position not only in qualitative fashion. Rather, the fit-monitoring unit 42 in the case determines quantitatively how strongly and in which direction the fit of the hearing instrument 4 deviates from the designated wearing position.

If the fit-monitoring unit 42 in the aforementioned embodiment determines a significant deviation of the sensor signal S from the reference value ascertained for the designated wearing position, said fit-monitoring unit causes (e.g., using one of the above-described methods) the output of a warning message to the user, which alerts the user to the incorrect fit of the hearing instrument 4 and contains instructions for correcting the fit.

Alternatively, the fit-monitoring unit 42 adjusts at least one signal processing parameter of the hearing instrument 4 on the basis of the sensor signal S output from the capacitive sensor 26 such that an effect of the mispositioning of the housing 8 is fully or partially compensated. For example, the fit-monitoring unit 42 adjusts a beamformer of the signal processor 20 of the hearing instrument 4 in such a way that a directional lobe of the beamformer is aligned with respect to the head of the user in the same way as in the designated wearing position (e.g., always perpendicular to the longitudinal axis of the head), even in the event of a mispositioned housing 8.

In a further refined embodiment of the invention, the fit-monitoring unit 42 follows a differentiated operational sequence of the method. In this case, the fit-monitoring unit 42 adapts the at least one signal processing parameter of the hearing instrument 4—as described above—to compensate for a detected misfit of the hearing instrument 4 only if the detected deviation of the fit of the hearing instrument 4 from the designated wearing position is small (in particular does not exceed a stored threshold value). Otherwise, i.e., in the event of more pronounced mispositioning of the hearing instrument 4, the fit-monitoring unit 42, as also described above, causes the output of the warning message, which alerts the user to the misfit of the hearing instrument 4 and contains an instruction for correcting the fit.

In a further embodiment of the invention, the fit-monitoring unit 42 contains a neural network which is trained on the user on an individual basis, contains the sensor signal S as an input signal and causes at least one corrective action in the event of a significant misfit of the hearing instrument 4, in particular (as described above) an adaptation of at least one signal processing parameter of the hearing instrument 4 to compensate for the misfit and/or the output of a warning message to the user.

In further embodiments of the invention, the hearing instrument is a conventional BTE device having a housing that is worn behind the ear and a receiver arranged in this housing. In this case, the connector 14 is formed by a sound tube. For example, a wire guided in the sound tube is used as sensor electrode 32 in this case. The statements made above as regards the embodiment and arrangement of the capacitive sensor and as regards checking the fit of the hearing instrument 4 on the ear can be easily transferred to this type of device.

The invention becomes particularly clear on the basis of the exemplary embodiments described above, but it is not restricted thereto by any means. Rather, further embodiments of the invention may be derived from the claims and the description above.

The following is a summary list of reference numerals and the corresponding structure used in the above description of the invention:
2 Hearing system
4 Hearing instrument
6 Hearing app
8 Housing
10 Earpiece
12 Receiver
14 Connector
16 Connecting cable
18 Microphone
20 Signal processor
22 Battery
24 (Wireless) communications apparatus
26 (Capacitive) sensor
28 Sensor controller
30 Sensor electrode
32 Sensor electrode
36 Smartphone
38 Shielding
40 Body structure
42 Fit-monitoring unit
A Response signal
E (Electric) field
I (Input) audio signal
M Ground
O (Output) audio signal
S Sensor signal
U_{B} Operating voltage
U_{S} Sensor voltage

1. A hearing instrument, comprising:
a housing to be worn in a designated wearing position behind an ear of a user;
an earpiece to be inserted into an auditory canal of the ear;
a flexible connector that connects said housing to said earpiece;
an output transducer for converting an output audio signal into a sound signal to be output to the user; and
a capacitive sensor having a control and evaluation circuit and two sensor electrodes electrically connected to said control and evaluation circuit;
wherein one of said two sensor electrodes is arranged in said housing and another of said two sensor electrodes is arranged in at least one of said connector or said earpiece.

2. The hearing instrument according to claim 1, wherein said control and evaluation circuit of said capacitive sensor is configured to apply an alternating voltage to one of said sensor electrodes and to measure a response signal responsive to the alternating voltage at the respectively other sensor electrode, the response signal being characteristic of an electrical capacitance of a capacitor formed by said sensor electrodes.

3. The hearing instrument according to claim 1, wherein:
said output transducer is arranged in said earpiece and said connector includes an electrical connecting cable for supplying the output audio signal to said output transducer, and wherein at least one of said connecting cable or said output transducer forms one of said sensor electrodes of said capacitive sensor.

4. The hearing instrument according to claim 1, wherein:
said output transducer is arranged in said housing and said connector is a hollow sound tube for supplying the sound signal created by said output transducer to said earpiece; and
at least one of said sensor electrodes of said capacitive sensor is arranged in said sound tube.

5. A hearing system, comprising:
a hearing instrument according to claim 1;
a fit-monitoring unit configured to perform at least one of the following:
check how the housing and the connector fit to the ear of the user on a basis of a sensor signal output by the capacitive sensor and, when the fit is determined as deviating from a designated wearing position, to output a message indicating the deviating fit; and/or
adjust at least one signal processing parameter of the hearing instrument on a basis of the capacitive sensor signal output by the capacitive sensor, in order to adapt a signal processing of the hearing instrument to the fit so determined.

6. A method of operating a hearing instrument, the method comprising:
providing a hearing instrument according to claim 1;
acquiring, by the capacitive sensor, a capacitive sensor signal as a measure of a fit of the housing and the connector on the ear of the user; and
when the fit is determined to deviate from the designated wearing position, outputting a message indicating that the fit deviates from the designated wearing position; and/or
adjusting at least one signal processing parameter of the hearing instrument on a basis of the capacitive sensor signal output by the capacitive sensor, in order to adapt the signal processing of the hearing instrument to the determined fit.
